(19) United States
(12) Patent Application Publication
(10) Pub. No.: US 2007/0012519 A1
(43) Pub. Date: Jan. 18, 2007

(54) System for merchandising disposable absorbent undergarments

(75) Inventor: Stephany Lyn Angielski, Neenah, WI (US)

Correspondence Address: KIMBERLY-CLARK WORLDWIDE, INC., 401 NORTH LAKE STREET, NEENAH, WI, 54956, US

(73) Assignee: Kimberly-Clark Worldwide, Inc.

(21) Appl. No.: 11/183,439

(22) Filed: Jul. 18, 2005

(51) Int. Cl. A61F 13/15; E04H 3/00

(52) U.S. Cl. 186/52

(57) ABSTRACT

The present invention provides a method or a system of providing disposable absorbent adult undergarments to a consumer in a retail store. The method or system has the steps of providing a package of reusable adult undergarments; providing a package of disposable absorbent adult undergarments; displaying the package of reusable adult undergarments in a first area of the retail store; and displaying the package of disposable absorbent adult undergarments in the first area of the retail store. The first area of the store may be an area which sells similar items, such as an aisle, a department, a rack, a shelving unit, a display and the like. In one embodiment of the present invention, the disposable absorbent adult undergarments are in visual proximity of the reusable adult undergarments. By providing the disposable absorbent adult undergarments in the same area of the store as the reusable adult undergarments, the purchaser and/or user of the disposable absorbent adult undergarments is provided with a shopping experience similar to those who are not in need of disposable absorbent adult undergarments. As result, the stigma or embarrassment often associated with purchasing disposable absorbent adult undergarments may be reduced, thereby providing a degree of normalcy for the purchaser and/or user of the disposable absorbent adult undergarments when a supply is to be purchased.

### FIELD OF THE INVENTION

The present invention relates generally to a system of providing disposable absorbent adult undergarments to a consumer in a retail store.

### BACKGROUND OF THE INVENTION

Disposable absorbent adult undergarments are currently displayed for sale in a retail store on an aisle with disposable incontinence pads and sanitary napkins. Typically disposable absorbent adult undergarments are packaged in bulk conventional packages, generally containing from about 15 to about 50 or more products. These disposable undergarments are generally packaged within the conventional package in either one or two rows of products, in a very similar manner in which diapers are currently packaged The packages generally sit on the store shelf and cannot be hung from merchandising apparatus.

Disposable absorbent adult undergarments have become an integral part of the lives of many people having a need for these disposable undergarments. These disposable undergarments contribute to normalcy of life for the user, by providing the user with a discrete means to deal with incontinence, bowel control and/or, in the case of females, menstruation. However, the normalcy of life for the user ends when the user must purchase or repurchase the disposable undergarments when his or her supply of the disposable undergarment runs out. That is, the user is forced to purchase additional disposable undergarments on an aisle of a retail store containing other incontinence pads, sanitary napkins, tampons and other personal care articles. As a result, it is often difficult for a user to purchase or repurchase a supply of these disposable undergarments in a private and discrete manner. This could lead to potential embarrassment, anxiety or be a traumatic experience for the purchaser, particularly if the purchaser happens to see someone known to the purchaser while the purchaser is in the aisle with the disposable undergarments.

There is a need to provide a discrete way for users of disposable absorbent adult undergarments to purchase a supply of the disposable absorbent adult undergarments without being forced to an aisle in a retail store which provides other disposable incontinence or disposable personal care articles. In addition, there is a need in the art to supply disposable absorbent adult undergarments to a user, which provides discretion and normalcy for the purchaser of disposable absorbent adult undergarments, by providing the user a means to purchase the disposable absorbent adult undergarments in a manner which is similar to the manner in which those who do not need disposable absorbent adult undergarments purchase their reusable undergarments.

Some users of adult disposable absorbent undergarments only need to use the disposable undergarments for a short period of time, for example, after surgery or after a life event which may adversely affect the mobility of the user. It can be difficult for the person in need of the disposable undergarment for the first time or on a temporary basis to locate the undergarment in a retail store. Therefore, there is a need in the art to provide a first time user or a temporary user of the disposable absorbent undergarment with a convenient way to locate the disposable absorbent undergarments.

Further, current disposable adult incontinence undergarments and feminine care undergarments are typically not provided to the consumer in a variety of styles. That is, currently available disposable undergarments are not provided in convention styles for durable (non-disposable) undergarments. For example, durable men's undergarments are provided in a variety of styles, such as boxers, briefs, boxer briefs, trunks and the like. Similarly, women's undergarments are provided in styles such as, bikini, brief, hi-cut, hipster and the like. As a result, the user is often dissatisfied with the fit of the disposable absorbent adult undergarment since they do not feel like reusable undergarments the user typically wore prior to needing the disposable absorbent adult undergarments.

As a result of the foregoing problems, users of disposable undergarments are often dissatisfied with both the fit of the disposable absorbent undergarments and difficulty in the selecting the appropriate disposable undergarments. This dissatisfaction is often due to the lack of normalcy in the methods in which the disposable absorbent undergarments are provided to the user in the retail store and/or the lack of selection. This is often exasperated by the current methods of providing disposable absorbent adult undergarments, which force users to go to a special area in a retail store set aside for disposable absorbent adult undergarments.

### SUMMARY OF THE INVENTION

Generally stated, the present invention provides a method or a system of providing disposable absorbent adult undergarments to a consumer in a retail store. The method or system has the steps of providing a package of reusable adult undergarments; providing a package of disposable absorbent adult undergarments; displaying the package of reusable adult undergarments in a first area of the retail store; and displaying the package of disposable absorbent adult undergarments in the first area of the retail store. The first area of the store may be an area which sells similar items, such as an aisle, a department, a rack, a shelving unit, a display and the like. In one embodiment of the present invention, the disposable absorbent adult undergarments are in visual proximity of the reusable adult undergarments. By providing the disposable absorbent adult undergarments in the same area of the store as the reusable adult undergarments, the purchaser and/or user of the disposable absorbent adult undergarments is provided with a shopping experience similar to those who are not in need of disposable absorbent adult undergarments. As a result, the stigma or embarrassment often associated with purchasing disposable absorbent adult undergarments may be reduced, thereby providing a degree of normalcy for the purchaser and/or user of the disposable absorbent adult undergarments when a supply is to be purchased. In addition, it can provide the additional benefit of aiding first time or temporary users of disposable absorbent adult undergarments to find the disposable absorbent adult undergarments in a retail store.

In another aspect of the present invention, the disposable absorbent adult undergarments are provided in two or more styles. In addition, the disposable absorbent adult undergarments may be gender specific. By providing the user with a variety of styles which fit differently, the user will be able to select a style of disposable absorbent adult undergarments which more closely resembles the style of reusable adult undergarments the user wore prior to needing the disposable absorbent adult undergarments. Styles of the disposable absorbent adult undergarments include for males, boxers, briefs, boxer briefs, and trunks, and for females, bikini, brief, hi-cut, and hipster. As a result, the transition from reusable adult undergarments to disposable absorbent adult undergarments is made easier for the user.

The present invention also provides a method or system of providing male specific and female specific disposable absorbent adult undergarments to a consumer or user of the disposable absorbent adult undergarments in a retail store in different areas of the retail store. This method includes the steps of providing a package of reusable adult male undergarments; providing a package of disposable absorbent adult male undergarments; providing a package of reusable adult female undergarments; providing a package of disposable absorbent adult female undergarments; displaying the package of reusable adult male undergarments in a first area of the retail store; displaying the package of disposable absorbent adult male undergarments in the first area of the retail store; displaying the package of reusable adult female undergarments in a second area of the retail store; and displaying the package of disposable absorbent adult female undergarments in the second area of the retail store.

The present invention further provides a method for instructing a retailer how to display or provide disposable absorbent adult undergarments to a consumer or user of the disposable absorbent adult undergarments in a retail store. This method has the steps of providing a package of disposable absorbent adult undergarments to a retailer; and instructing the retailer to display the package of disposable absorbent adult undergarments in a first area of the retail store that displays reusable undergarments.

DEFINITIONS

It should be noted that, when employed in the present disclosure, the terms “comprises”, “comprising” and other derivatives from the root term “comprise” are intended to be open-ended terms that specify the presence of any stated features, elements, integers, steps, or components, and are not intended to preclude the presence or addition of one or more other features, elements, integers, steps, components, or groups thereof.

As used herein, the term “disposable” refers to articles which are designed to be discarded after a limited use rather than being laundered or otherwise restored for reuse.

As used herein, the term “disposable absorbent adult undergarment” refers to an article which is capable of absorbing bodily fluids which resembles undergarments, such as women's panties and men's underwear. These articles are intended to be discarded when they become soiled with bodily fluids, such as urine, bowel movements and menstrual fluid.

“Retail Store”, refers to a business that offers durable and/or disposable products for sale to a consumer or purchaser. As used herein, the term “retail store” is intended to include department stores, grocery stores, drug stores, clothing stores, discount stores, warehouse club stores and the like.

“Reusable” refers to an item intended to be used multiple times which may be restored to near the original state by a process such as laundering. A reusable item is not considered to be disposable after an initial use. Reusable items are sometimes referred to as durable. Generally, reusable undergarments are washable before reuse.

“Reusable adult undergarment” as used herein, refers to an article of clothing typically worn under other articles of clothing, which is intended to be used multiple times before discarding. Examples include men's underwear and women's panties.

“Spatially separated” refers to regions or items in a retail store that are not in proximity to one another, such as might require a consumer to travel to a remote or different region of the store, or to exit one region such as an aisle, to reach the other region or item; adjoining regions of the store, such as on either side (i.e., facing sides) of an aisle, are not considered to be spatially separated; however, consecutive aisles would generally be considered to be spatially separated. Regions or items in a retail store that are spatially separated are not in visual proximity to one another.

As used herein, the term “undergarments” is intended to mean items of clothing intended to be worn under other items of clothing. Undergarments include underwear, panties and the like.

Visual proximity” refers to areas or items in a retail store within 15 meters of one another and that can be viewed from a consumer's location in the retail store by the turning of the consumer's head or by the turning around of the consumer's body.

### DETAILED DESCRIPTION OF THE INVENTION

The present invention provides a method or a system of providing disposable absorbent adult undergarments to a consumer in a retail store. The method or system has the steps of providing a package of reusable adult undergarments; providing a package of disposable absorbent adult undergarments; displaying the package of reusable adult undergarments in a first area of the retail store; and displaying the package of disposable absorbent adult undergarments in the first area of the retail store.

In another method of the present invention, provided is a method for instructing a retailer how to display or provide disposable absorbent adult undergarments to a consumer or user of the disposable absorbent adult undergarments in a retail store. This method has the steps of providing a package of disposable absorbent adult undergarments to a retailer; and instructing the retailer to display the package of disposable absorbent adult undergarments in a first area of the retail store that displays reusable undergarments. As is set forth in this portion of the invention, the instructing step includes directing, requiring or suggesting a retailer provide the disposable absorbent undergarments to a consumer or user in an area of the retail store which provides reusable undergarments to a consumer.

The area of a retail store aisle can be an area in which the retail store sells gender specific clothing, an aisle of the retail store, a store rack, a shelving unit or a store display, depending on the type of retail store. Generally, disposable absorbent adult undergarments should be displayed or merchandised in the visual proximity of the reusable undergarments. As is defined above, visual proximity means that the area or items displayed are within 15 meters of one another which can be viewed from a consumer's location in the retail store by rotation of the consumer's head or by rotation of the consumer's body. Desirably, the items are displayed within 10 meters of one another and more desirably within 5 meters of one other. Most desirably, the items are displayed within 3 meters of one another.

In the present invention, the disposable absorbent adult undergarments are desirably displayed in the first area of a retail store with the reusable undergarments. This first area may be separate and distinct from another area of the retail store that displays and merchandizes absorbent personal care articles, such as disposable incontinence pads and sanitary napkins, tampons and the like. For example, the first area of the retail store may display or merchandize men's clothing, including reusable men's undergarments. In the present invention, adult men's disposable absorbent undergarments are also provided in this area. In another area of the retail store, the retail store displays or merchandizes absorbent personal care products. Likewise, the first area of the retail store may display or merchandize women's clothing, including reusable women's undergarments. In the present invention, adult women's disposable absorbent undergarments are also provided in this area. In another area of the retail store, the retail store displays or merchandizes absorbent personal care products. The personal care products located in the other area of the retail store may also include disposable absorbent adult undergarments, in addition to other disposable absorbent personal care products. Stated another way, it is possible to display and merchandize the disposable absorbent adult undergarments in a single area of the retail store, with the reusable undergarments or the disposable absorbent adult undergarments may also be displayed and mechanized in at least one additional area of the retail store, which provides absorbent personal care articles.

The disposable absorbent adult undergarments can be gender specific, i.e. designed for males or females. In the case of male specific disposable absorbent adult undergarments, they may be provided in a variety of styles, which are common with reusable adult male undergarments, including, for example, briefs, boxers, boxer trunks, or trunks. Desirably, the disposable absorbent adult male undergarments are provided in two or more styles. Similarly, in the case of female specific disposable absorbent adult undergarments, the adult female undergarments may be provided in conventional styles in which reusable adult female undergarments are currently provided, such as bikini, brief, hipster, or hi-cut. Likewise, it is desirable that the female disposable absorbent adult undergarments are provided in two or more styles. In addition to incontinence type undergarments designed specifically for females, the absorbent undergarments may also be absorbent undergarments which are specifically designed for feminine care, i.e. are designed to absorb and contain menstrual fluid.

By providing a variety of styles of disposable absorbent adult undergarments to the users of these articles, the transition from reusable adult undergarments to disposable absorbent adult undergarments is made easier for the user. Disposable absorbent adult undergarments typically are only provided in a single style, and only vary by size. In addition, disposable absorbent adult undergarments are also generally provided in a brief style. As a result of having a lack of styles for a user to select from, it is often difficult, if not impossible, for an user to become comfortable wearing the disposable absorbent adult undergarment.

The adult male disposable absorbent undergarments are provided in various sizes. Typical sizes are, for example, small, medium, large, and x-large and the like. In addition to providing these sizes, the package may also have a conversion which equates a particular size marked on the package to a waist size or a range of waist sizes. By providing this conversion, the users are able to select the appropriate size. The conversion may be provided in a chart on the package, or the conversion may be placed next to the size designation. For example, a designation could be Large (38) or Large (38-40). Alternatively, the male disposable absorbent adult undergarments could also be provided in ranges of waist sizes, without the small, medium, large, and x-large size designations.

Similarly, the adult female disposable absorbent adult undergarments are provided in various sizes. Typical sizes are, for example, small, medium, large, and x-large and the like. In addition to providing these sizes, the package may also have a conversion which equates a particular size marked on the package to sizes conventionally marked on women's undergarments or a range of sizes, which is generally even numbers starting with 2, for example, 2, 4, 6, 8 etc. However, some manufacturers use odd numbers starting with 1, for example 1, 3, 5, 7 etc. Others provide undergarment sizing with a continuous numbers, for example, 1, 2, 3, 4, 5 etc. For example, a designation could be Small (2) or Small (2-4). Alternatively, the female disposable absorbent adult undergarments could also be provided in ranges of conventional sizes, without the small, medium, large, and x-large size designations. Sizing could also be set-up with other methods, such as by waist or hip size, by height and weight or by other known methods used to provide sizing on women's clothing items.

The present invention also provides a method or system of providing male specific and female specific disposable absorbent adult undergarments to a consumer or user of the disposable absorbent adult undergarments in a retail store in different areas of the retail store. This method includes the steps of providing a package of reusable adult male undergarments; providing a package of disposable absorbent adult male undergarments; providing a package of reusable adult female undergarments; providing a package of disposable absorbent adult female undergarments; displaying the package of reusable adult male undergarments in a first area of the retail store; displaying the package of disposable absorbent adult male undergarments in the first area of the retail store; displaying the package of reusable adult female undergarments in a second area of the retail store; and displaying the package of disposable absorbent adult female undergarments in the second area of the retail store.

In this method, the first area of the store may display and provide for sale to customers adult male clothing and the second area of the store may display and provide for sale to customers adult female clothing. By having the clothing separated by gender, the disposable absorbent adult undergarments can be purchased by consumers in need of disposable absorbent adult undergarments in essentially the same way as reusable undergarments are currently purchased.

The displaying of the package of reusable adult male undergarments in the first area and the displaying of the package of disposable absorbent adult male undergarments in the first area is such that the package of disposable absorbent adult male undergarments is visually proximate the package of reusable adult male undergarments. Likewise, the displaying packages of reusable adult female undergarments in the second area and the displaying packages of disposable absorbent adult female undergarments in the second area is such that the package of disposable absorbent adult female undergarments is visually proximate the package of reusable adult female undergarments. As is defined above, visual proximity means that the area or items displayed are within 15 meters of one another which can be viewed from a consumer's location in the retail store by rotation of the consumer's head or by rotation of the consumer's body. Desirably, the items are displayed within 10 meters of one another and more desirably within 5 meters of one other. Most desirably, the items are displayed within 3 meters of one another. Ideally, the disposable absorbent adult undergarments should be displayed on the same aisle of the retail store, the same store rack, the same shelving unit or the same store display, depending on the type of retail store, as the reusable undergarments.

Typically, disposable absorbent adult undergarments are provided in packages containing about 5 to about 20 disposable absorbent adult undergarments. One thing that may present problems for retailers is that disposable absorbent adult undergarments tend to be fairly bulky and take a fairly large amount of space in a retail store. Therefore, providing the packages of disposable absorbent adult undergarments in the current packaging means may make it difficult to display the disposable absorbent adult undergarments with the reusable undergarments. Desirably, the size of the packages with the disposable absorbent adult undergarments should be generally about the same size or a little larger than the packages of the reusable undergarments. To reduce the size of the packaging, it is possible to highly compress the disposable absorbent adult undergarments within the packaging. Another alternative would be to vacuum pack the packages. Yet another method to reduce the size of the packages is to reduce the number of disposable absorbent adult undergarments in a given package; however, this is less preferred since the packages may not provided a user with a sufficient number of disposable absorbent adult undergarments. In addition, the shape of the packaging should be approximately the same shape as the packaging for reusable underwear. The packaging for the disposable absorbent adult undergarments can also be provided with means to hang on a rack, such as providing a hole in the packaging to allow the package of disposable absorbent adult undergarments to be placed on a peg, pin or other type of hanger typically found in retail stores. Generally, hanging bags have an extended top portion which have a hole that allows the package to hang on a peg, pin or other type of hanger.

Ideally, in the present invention, the disposable absorbent adult undergarments are displayed or merchandized in a way such that the disposable absorbent adult undergarments are intermingled among the reusable undergarments. In addition, the current areas of the retail store which sell disposable absorbent adult undergarments along with other absorbent articles may still be provided or, the disposable absorbent adult undergarments may be moved to the area of the store which sells reusable undergarments and not be provided in the area of the store which sells other disposable products, such as sanitary napkins, tampons and the like.

As a result of the present invention, consumers who use disposable absorbent adult undergarments are provided with a shopping experience which is similar, if not identical to purchasing reusable undergarments. Selection of the disposable absorbent adult undergarments would be essentially similar to the selection of reusable undergarments, by providing the consumer with different styles to choose from, or providing the disposable absorbent adult undergarments in sizes the user would normally purchase reusable underwear. In addition, by providing the disposable absorbent adult undergarments in a section or area of a retail store which also displays and merchandizes reusable undergarments, the user is provided with a way to purchase disposable absorbent adult undergarments without having the go to the area of the retail store that typically sells disposable absorbent adult undergarments. Often potential embarrassment, anxiety or otherwise traumatic experience for the purchaser may occur when purchasing disposable absorbent adult undergarments from the absorbent article aisle, reminding the user that they have a problem and that their lives are not within the norm. This potential embarrassment, anxiety or traumatic experience for the purchaser may be avoided using the process or system of the present invention.

Other ways of providing the disposable absorbent adult undergarments to a consumer or user in a retail store within the scope of the present invention, include, for example, providing both a reusable undergarment and disposable absorbent adult undergarment in a single package or co-packing disposable absorbent adult undergarment in a single package. Co-packing could be accomplished by co-branding the disposable absorbent adult undergarment with the tradename of the disposable absorbent adult undergarments and the brand name of the reusable undergarmets, by branding the disposable adult undergarments with the brand name of the reusable undergarments, or by branding the reusable undergarments with the brand name the disposable absorbent undergarments. It is also possible in the present invention to co-pack other personal care articles, such as razors, aftershave, deodorants, cosmetics, skin care/body care, perfumes and the like.

Those skilled in the art will recognize that the present invention is capable of many modifications and variations without departing from the scope thereof. Accordingly, the detailed description and examples set forth above are meant to be illustrative only and are not intended to limit, in any manner, the scope of the invention as set forth in the appended claims.

1. A method of providing disposable absorbent adult undergarments to a consumer or user of the disposable absorbent adult undergarments in a retail store, said method comprising:
providing a package of reusable adult undergarments;
providing a package of disposable absorbent adult undergarments;
displaying the package of reusable adult undergarments in a first-area of the retail store; and
displaying the package of disposable absorbent adult undergarments in the first area of the retail store.

2. The method of claim 1, wherein the displaying of the package of reusable adult undergarments in the first area and the displaying of the package of disposable absorbent adult undergarments in the first area is such that the package of disposable absorbent adult undergarments are within the visual proximity of the package of reusable adult undergarment.

3. The method of claim 1, wherein the disposable absorbent adult undergarments comprise disposable absorbent adult incontinence undergarment or disposable absorbent feminine care menstrual undergarments.

4. The method of claim 1, wherein the first area comprises an aisle of a retail store.

5. The method of claim 3, wherein the first area comprises a store rack, a shelving unit or a store display.

6. The method of claim 1, wherein the disposable absorbent adult undergarments comprise adult male disposable absorbent undergarments and the reusable undergarments comprise adult male reusable undergarments.

7. The method of claim 6, wherein in the area of the store displays and provides adult male clothing for sale to a customer.

8. The method of claim 7, wherein the disposable absorbent adult undergarments are provided in two or more styles.

9. The method of claim 8, wherein the styles comprise briefs, boxers, boxer trunks, or trunks.

10. The method of claim 6, wherein the adult male disposable absorbent undergarments are provided in various sizes comprising small, medium, large, and x-large.

11. The method of claim 10, wherein the package of adult male disposable absorbent undergarments comprises a conversion from a size marked on the package to a waist size.

12. The method of claim 1, wherein the disposable absorbent adult undergarments comprise adult female disposable absorbent undergarments and the reusable undergarments comprise adult female reusable undergarments.

13. The method of claim 12, wherein in the area of the store displays and provides adult female clothing for sale to a customer.

14. The method of claim 13, wherein the disposable absorbent adult undergarments are provided in two or more styles.

15. The method of claim 14, wherein the style comprises bikini, brief, hipster, or hi-cut.

16. The method of claim 12, wherein the disposable absorbent undergarments are provided in various sizes comprising small, medium, large, and x-large.

17. The method of claim 16, wherein the package of adult female disposable absorbent undergarments comprises a conversion from a size marked on the package to sizes conventionally used to size reusable female undergarments.

18. A method of providing disposable absorbent adult undergarments to a consumer or user of the disposable absorbent adult undergarments in a retail store, said method comprising:
providing a package of reusable adult male undergarments;
providing a package of disposable absorbent adult male undergarments;
providing a package of reusable adult female undergarments;
providing a package of disposable absorbent adult female undergarments;
displaying the package of reusable adult male undergarments in a first area of the retail store; and
displaying the package of disposable absorbent adult male undergarments in the first area of the retail store;
displaying the package of reusable adult female undergarments in a second area of the retail store; and
displaying the package of disposable absorbent adult female undergarments in the second area of the retail store.

19. The method of claim 18, wherein the first area of the store displays and provides for sale to a customer adult male clothing and the second area of the store displays and provides for sale to a customer adult female clothing.

20. The method of claim 18, wherein the displaying of the package of reusable adult male undergarments in the first area and the displaying of the package of disposable absorbent adult male undergarments in the first area is such that the package of disposable absorbent adult male undergarments is visually proximate the package of reusable adult male undergarments and wherein the displaying of the package of reusable adult female undergarments in the second area and the displaying of the package of disposable absorbent adult female undergarments in the second area is such that the package of disposable absorbent adult female undergarments is visually proximate the package of reusable adult female undergarments.

21. A method of directing a retailer to provide disposable absorbent adult undergarments to a consumer or user of the disposable absorbent adult undergarments in a retail store, said method comprising:
providing a package of disposable absorbent adult undergarments to a retailer; and
instructing the retailer to display the package of disposable absorbent adult undergarments in a first area of the retail store that displays reusable undergarments.

22. The method of claim 21, wherein the first area comprises an aisle of a retail store.

23. The method of claim 22, wherein the first area comprises a store rack, a shelving unit or a store display.

24. The method of claim 21, wherein said instructing comprises suggesting to the retailer that the disposable absorbent adult undergarments should be displayed with the reusable undergarments.
